(19) United States
(12) Patent Application Publication
(10) Pub. No.: US 2013/0039993 A1
(43) Pub. Date: Feb. 14, 2013

(54) ARGON-BASED INHALABLE GASEOUS MEDICAMENT AGAINST PERIPHERAL ORGAN DEFICIENCIES OR FAILURES

(71) Applicant: Marc Lemaire, Paris (FR)

(72) Inventor: Marc Lemaire, Paris (FR)

(73) Assignee: L'Air Liquide, Societe Anonyme pour l'Etude et l'Exploitation des Procedes Georges Claude, Paris (FR)

(21) Appl. No.: 13/576,242

(22) PCT Filed: Jan. 21, 2011
(86) PCT No.: PCT/FR2011/050111

§ 371 Date: Oct. 11, 2012

(30) Foreign Application Priority Data
Feb. 15, 2010 (FR) 1051040

(51) Int. Cl. A61K 33/00; A61P 9/00; C01B 23/00; A61P 11/00; A61P 1/16; A61P 13/12

(52) U.S. Cl. 424/600; 423/262

(57) ABSTRACT

The invention relates to a gaseous composition containing an effective amount of argon gas for use, by inhalation, in preventing or treating a deficiency or failure of at least one peripheral organ in a patient. Preferably, it contains between 15 and 80% by volume of argon, and oxygen, preferably at least 21% by volume of oxygen. The organ deficiency or failure thus treated is transient, preferably from less than one hour to a plurality of days or weeks, or definitive. The organ concerned may be the liver, the kidneys or the lungs.

### CROSS REFERENCE TO RELATED APPLICATIONS

This application is a 371 of international PCT Application PCT/FR2011/050111, filed Jan. 21, 2011, which claims priority to French Application 1051040, filed Feb. 15, 2010, the entire contents of which are incorporated herein by reference.

### BACKGROUND

The invention relates to the use of argon gas for producing all or part of an inhalable medicament intended for treating or preventing a deficiency or a failure of a peripheral organ chosen from the kidneys, the liver and the lungs, whether this deficiency is isolated or multiple.

The deficiency or failure of an organ is characterized by an organic and/or functional abnormality of the organ under consideration, i.e. an inability of this organ to exhibit a normal functionality or the impossibility of it exhibiting a normal functionality.

This deficiency or failure may be transient, i.e. may last from less than one hour to several days or weeks, or may be definitive.

The cause of an organ deficiency or failure may be connected in particular to a vascularization abnormality/defect, for example ischemia/reperfusion, or an oxygenation abnormality/defect, a localized or systemic infliction, a localized or systemic, acute or chronic inflammation, primary or secondary autoimmunity phenomena, a trauma, an organo-degeneration, etc.

The deficiency or failure may concomitantly affect several organs, and the term multiorgan deficiency is then used.

It is known to be able to treat certain deficiencies or failure affecting the brain by means of inhaled gases. Thus, document EP-A-1158992 proposes using inhaled xenon for combating cerebral, in particular ischemic, neuro-intoxications which are characterized by a cerebral dysfunction of one or more neurotransmission systems.

Moreover, document EP-A-1541156, for its part, proposes using inhaled argon for combating said cerebral neuro-intoxications. The neuroprotective potential of argon gas, administered alone or as a mixture with nitrous oxide, on the development and expression of sensitization to D-amphetamine has also been evaluated.

However, the mechanisms of action of argon are still poorly understood and could be based on a GABA_{A}-receptor, in particular benzodiazepine-site, agonist action as described by J. Abraini at al., Anesth Analg, 2003, 96:746-749.

Recently, the neuroprotective capacity of argon has been confirmed by reference Loetscher et al., Critical Care, 2009, 13 :R206 ;doi:10.1186/cc8214.

Argon has been shown to be able to protect cochlear cells in isolated culture, i.e. the “hair” cells derived from the organ of Corti of the rat, as described by Yarin et al., Hear Res 2005, 201:1-9. These cells can be likened to cerebral neurons, for which other gases, in particular xenon, have been shown to be able to exhibit a neuroprotective effect, as recalled by Ma et al., Br J Anaesth, 2002, vol 98: 739-46.

In addition, document EP-A-1651243 for its part, proposes using mixtures of Xe/N₂O inhaled for this purpose, while document DE-A-19991033704 proposes liquid formulations containing dissolved xenon for treating cerebral ischemia and hypoxia.

As is seen, most of the proposed treatments are aimed at treating essentially cerebral deficiencies or failure, i.e. those which affect only the brain.

However, protecting only the brain is not sufficient. Indeed, it is advisable to also be able to protect the peripheral organs against any failure, in particular the vital organs such as the heart, the liver, the kidneys, etc.

Recent studies have shown that xenon can have an ability to protect organs other than the brain against the consequences of ischemia/reperfusion syndromes, for example to protect the kidney, as described by reference Ma at al., J Am Soc Nephrol 2009, 20-4 :713-20, or the heart, as taught by Schweibert at al., Eur J Anaesth 2010, Epub.

Moreover, helium, neon and argon are described by P. Pagel, in Cardioprotection by Noble Gases, J. of Cardioth. & Vasc. Anaesth., vol. 24, n° 1, 2010, as having cardiac protective properties, where they are administered at a content of 70% by volume, said properties being independent of the anesthetic effect.

In addition, document DE-A-10319837 teaches the use of argon, neon, helium, krypton, xenon or mixtures thereof for therapeutic or prophylactic purposes against inner ear conditions, in particular otitis, noise-induced injuries, etc.

Currently however, there is no really effective medicament for protecting one or more peripheral organs, other than the brain or the heart, against a deficiency characterized by an organic or functional abnormality of the organ under consideration, i.e. an inability of this organ to exhibit a normal functionality or the impossibility of it exhibiting a normal functionality, in particular of a peripheral organ such as the kidneys, the liver and the lungs, regardless of the cause thereof.

The problem is therefore that of proposing a preventive and/or curative treatment for a deficiency or failure of one (or more) peripheral organ(s) chosen from the kidneys, the liver and the lungs, regardless of the cause thereof.

### SUMMARY

The solution of the invention is therefore a gaseous composition containing an effective amount of argon gas for use, by inhalation, in preventing or treating a deficiency or failure of at least one peripheral organ in a patient, said peripheral organ being chosen from the liver, the kidneys and the lungs.

Depending on the case, the inhalable gaseous composition or gaseous medicament of the invention may comprise one or more of the following characteristics:
it contains between 15 and 80% by volume of argon;
it contains at least 30% by volume of argon;
it contains less than 75% by volume of argon;
the organ deficiency results from an organic and/or functional abnormality;
the organ deficiency concerns several organs;
the organ deficiency is transient, preferably from less than one hour to several days or weeks, or is definitive;
the organ deficiency concerns several organs comprising one or more organs chosen from the liver, the kidneys and the lungs and, moreover, the heart;
said compositional medicament also contains oxygen, preferably at least 21% by volume of oxygen;
it also contains an additional compound chosen from the group made up of N₂O, Xe, He, Ne, NO, CO, H₂S and N₂;
the argon is administered to the patient one or more times a day for an inhalation period of from a few minutes to one or more hours;
said compositional medicament is made up of a gas mixture containing, in addition, oxygen, nitrogen or mixtures thereof, in particular air;
it is made up of a binary gas mixture consisting of argon and of oxygen for the rest, or a ternary mixture consisting of argon, nitrogen and oxygen; the gaseous medicament is preferably ready to use;
it is packaged in a gas bottle.

The invention also relates to the use of argon gas for producing an inhalable medicinal composition according to the invention intended for preventing or treating a deficiency or failure of at least one peripheral organ in a patient, said at least one peripheral organ being chosen from the liver, the kidneys and the lungs.

Generally, during treatment, the argon is administered to the patient by inhalation, for example by means of a ventilator, or a nebulizer, or spontaneously with prepackaged gas bottles connected to a face mask or nasal mask, or nasal goggles.

The duration of administration depends on the duration of the deficiency/failure or of the risk of failure, chosen case by case affecting the patient under consideration; for example, the argon can be administered for an administration period of from a few minutes to a few tens of minutes, or even of hours, for example less than one hour, at a frequency that can reach one or more times a day and over a total treatment period of one or more days, weeks, months or years.

The argon or argon-based gas mixture is preferentially packaged in a gas bottle under pressure or in liquid form, for example in a bottle of one or more liters (water content) and at a pressure of between 2 and 300 bar.

The argon or argon-based gas mixture can be in “ready-to-use” form, for example premixed with oxygen, or it can be mixed on site at the time of use, in particular with oxygen and optionally with another gaseous compound, for example nitrogen.

In the context of the invention, the patient is a human being, i.e. a man or a woman, including children, adolescents or any other group of individuals, for example newborn babies.

### Example

The following tests were carried out in order to demonstrate that argon has a protective effect on the preservation of a peripheral organ, namely in this case a kidney.

To do this, kidneys isolated from rats intended for a transplantation were used.

In this study, the effects of the argon gas were studied on the functional and morphological characteristics of the kidneys isolated.

120 Wistar rats, weighing between 200 g and 250 g, were used while adhering to the Guide for the Care and Use of Laboratory Animals published by the National Institutes of Health.

The animals were operated upon under general anesthesia and the left kidneys were removed and then preserved in several gaseous atmospheres, namely:
Group A (according to the invention): argon (100% by volume)
Group B (comparative): xenon (100% by volume)
Group C (1st control): air (i.e. essentially N₂/O₂ mixture)
Group D (2nd control): nitrogen

The kidneys of a rat are preserved therein for 6 hours before being grafted onto another anesthetized rat according to the conditions previously described. The rats are kept under observation for 14 days.

In order to carry out an evaluation of the effectiveness of the protection produced by the various gases tested, the renal function and renal morphology of the grafted kidneys were examined.

The renal function was evaluated by calculating the creatinine clearance estimated and related back to 100 g of weight, and by measuring the 24-hour albuminuria.

The renal morphology was evaluated by standard histology and histochemistry using anti-active caspase-3 and anti-CD10 antibodies.

### BRIEF DESCRIPTION OF THE DRAWINGS

The results obtained regarding the renal function are shown diagrammatically in FIGS. 1 and 2, which represent:

FIG. 1: creatinine clearance at days D7 and D14

FIG. 2: albuminuria at days D7 and D14.

### DESCRIPTION OF PREFERRED EMBODIMENTS

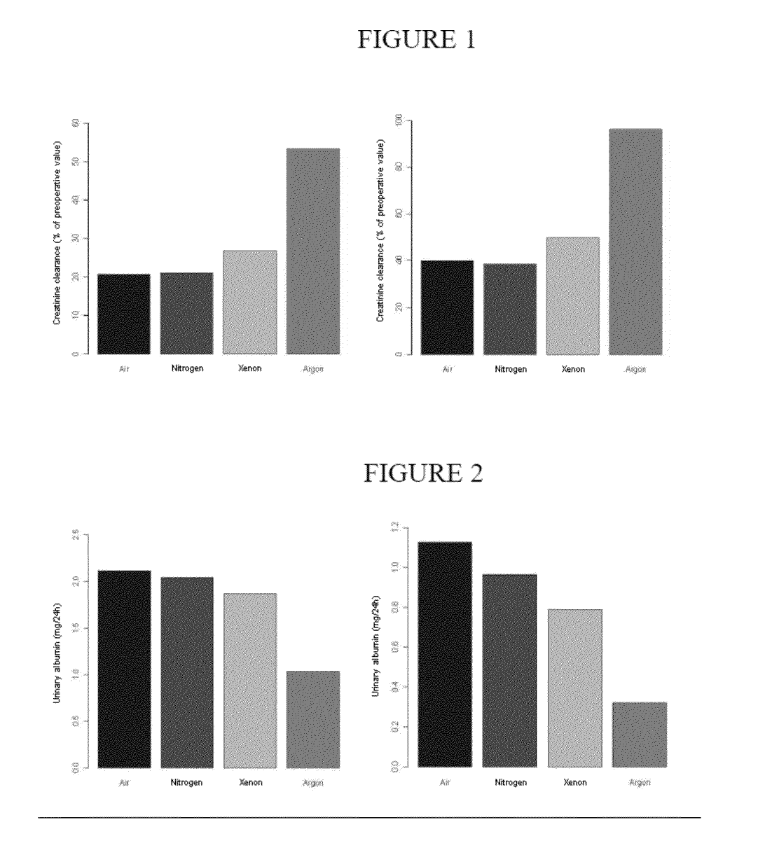

As is noted, FIG. 1 clearly shows that argon is particularly effective in that it preserved a good creatinine clearance of the grafted kidneys of group A. The creatinine clearance measurement is the main biological parameter for a renal function evaluation.

Conversely, for the groups B, C and D, the creatinine clearance is very low for the groups treated with nitrogen and air, and slightly better with xenon, but significantly lower compared with argon. Thus, the excretion function of the kidney is preserved under optimum conditions with argon.

Moreover, FIG. 2 shows that, in the argon group, albuminuria (presence of albumin in the urine which normally does not contain any, and which indicates considerable destruction of the glomerular structure of the kidney) is half as much in group A than in the other groups, in particular compared with nitrogen and with air.

This demonstrates that argon protects the structure of the kidney, which will be confirmed by the rest of the experiment (histology).

With regard to the renal morphology evaluated by standard histology, the kidneys of control groups C and D (air and nitrogen) show cell lysis with subconfluent coagulation necrosis.

Close to ⅓ of the kidneys of group B (xenon) exhibit injuries of the same type, but no kidney of group A under an argon atmosphere exhibited any sign of cell lysis or necrosis.

Furthermore, with regard to the renal morphology evaluated by histochemistry, the organs of group A (Ar according to the invention) appear to be better preserved than those of the other groups. The CD10 labeling is very well preserved, which amounts to preservation of the tubule functional structure, whereas the caspase-3 labeling is discrete and in foci, thereby showing very reduced cell degeneration, unlike what is found in the xenon group (group B) and especially the control groups (groups C and D).

Finally, the kidneys preserved under argon in accordance with the invention retain their functional capacities, and also their physical integrity (in the cell and tissue sense) since the argon reduces ischemic/reperfusion injuries and improves renal function.

The mechanism of action via which argon is capable of protecting these organs, in particular kidney, heart, liver and lungs, during an isolated or multiple, acute or chronic deficiency characterized by a transient or definitive, organic and/or functional abnormality, has not yet been established with certainty. However, it may be imagined that argon could act through maintaining effective homeostasis, oxygenation, vascularization and/or regulation of the mechanisms of cell apoptosis, i.e. the mechanisms that result in cell death, etc.

In any event, these tests show that argon can be used as an inhalable medicament for preventing or treating a deficiency or failure of one or more peripheral organs in a patient, in particular the liver, the kidneys and the lungs, whether the organ failure is due to a localized or systemic infection, a localized or systemic, acute or chronic inflammation, primary or secondary autoimmunity phenomena, a trauma, an organo-degeneration or another cause.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

1. A gaseous composition comprising an effective amount of argon gas capable of preventing or treating a deficiency or failure of at least one peripheral organ in a patient, said at least one peripheral organ being chosen from the liver and the kidneys upon inhalation by a patient, wherein the gaseous composition is suitable for administration to the patient by inhalation.

2. The composition of claim 1, wherein the gaseous composition comprises between 15 and 80% by volume of argon.

3. The composition of claim 1, wherein the gaseous composition comprises at least 30% by volume of argon.

4. The composition of claim 1, wherein the gaseous composition comprises less than 75% by volume of argon.

5. The composition of claim 1, wherein the organ deficiency or failure results from an organic and/or functional abnormality.

6. The composition of claim 1, wherein the organ deficiency or failure concerns several organs.

7. The composition of claim 1, wherein the organ deficiency or failure is from less than one hour to more than one day.

8. The composition of claim 1, wherein the organ deficiency or failure further comprises at least one other organ being chosen from the heart and the lungs.

9. The composition of claim 1, wherein the gaseous composition comprises oxygen.

10. The composition of claim 1, wherein the gaseous composition comprises an additional compound chosen from the group made up of N₂O, Xe, He, Ne, NO, CO, H₂S and N₂.

11. The composition of claim 9, wherein the oxygen is at least 21% (by volume) of the gaseous composition.

12. A method of preventing or treating a deficiency or failure of at least one peripheral organ in a patient, said at least one peripheral organ being chosen from the liver and the kidney, the method comprising the step of administering a gaseous composition to a patient by inhalation, wherein the gaseous composition comprises an effective amount of argon gas capable of preventing or treating a deficiency or failure of at least one of the liver or the kidney, to thereby prevent or treat the deficiency or failure of the liver or the kidney or both.

13. The method of claim 1, wherein the gaseous composition comprises between 15 and 80% by volume of argon.

14. The method of claim 1, wherein the gaseous composition comprises at least 30% by volume of argon.

15. The method of claim 1, wherein the gaseous composition comprises less than 75% by volume of argon.

16. The method of claim 1, wherein the organ deficiency or failure results from an organic and/or functional abnormality.

17. The method of claim 1, wherein the organ deficiency or failure concerns several organs.

18. The method of claim 1, wherein the organ deficiency or failure is from less than one hour to more than one day.

19. The method of claim 1, wherein the organ deficiency or failure further comprises at least one other organ being chosen from the heart and the lungs.

20. The method of claim 1, wherein the gaseous composition comprises oxygen.

21. The method of claim 12, wherein the gaseous composition comprises an additional compound chosen from the group made up of N₂O, Xe, He, Ne, NO, CO, H₂S and N₂.

22. The method of claim 20, wherein the oxygen is at least 21% (by volume) of the gaseous composition.
